(19) United States
(12) Patent Application Publication
(10) Pub. No.: US 2017/0113066 A1
(43) Pub. Date: Apr. 27, 2017

(54) SYSTEM TO PRODUCE ANATOMICAL REPRODUCIBILITY AND DETECT MOTION DURING A MEDICAL TREATMENT AND METHODS OF USE

(71) Applicant: FUNDAÇÃO D. ANNA DE SOMMER CHAMPALIMAUD E DR. CARLOS MONTEZ CHAMPALIMAUD, LISBOA (PT)

(72) Inventor: Carlo GRECO, Lisbon (PT)

(21) Appl. No.: 15/301,800

(22) PCT Filed: Apr. 2, 2015
(86) PCT No.: PCT/EP2015/057333

§ 371 Date: Oct. 4, 2016

(30) Foreign Application Priority Data
Apr. 4, 2014 (PT) 107570

(51) Int. Cl. A61N 5/10; A61B 90/00

(52) U.S. Cl. CPC A61N 5/1049; A61N 5/1067; A61B 90/39; A61N 2005/1051; A61N 2005/1055; A61N 2005/1097; A61B 2090/3966; A61M 25/04

(57) ABSTRACT

A system that produces anatomical reproducibility during a medical procedure includes a catheter configured to produce anatomical reproducibility and to detect motion of target tissue, which includes a catheter that has a first lumen formed therein. The motion tracking device includes a motion tracking assembly that is removably disposed within the first lumen. The motion tracking assembly has a plurality of motion tracking elements that are connected to one another with a connector. The motion tracking elements are spaced apart at fixed distances relative to one another. The motion tracking elements are configured such that movement of any one of the motion tracking elements is detected by the motion tracking device, thereby indicating that the target tissue has moved during the medical procedure. Detecting such movement in real-time allows the operator (e.g., radiation oncologist) to take any necessary immediate remedial action including stopping the medical procedure.

### TECHNICAL FIELD

The present invention is generally directed to the field of medical procedures (medical treatments) and in particular is directed to a system that produces anatomical reproducibility during a medical treatment and includes as a part thereof a non-implantable, removable motion tracking device for use in the medical treatment (e.g., non-invasive procedures, such as ablative high-dose radiotherapy) that detects whether the target tissue remains stable in a target position during the entire medical procedure.

### BACKGROUND

In many types of medical treatments, it is critical that the target tissue, such as an organ or the like, remains in a stable position (i.e., in an anatomically reproducible state) during the entire medical procedure to ensure that the proper tissue is being subjected to the treatment. This is especially true for radiotherapy in which radiation is delivered to the target tissue since the radiation is toxic to surrounding healthy tissue.

Radiotherapy is one type of treatment for cancer, such as prostate cancer, cervical cancer, rectal cancer, etc.

In particular and with respect to prostate cancer, external beam radiation therapy given with conventional fractionated schedules to a total dose of 78-86 Gy is an effective definitive treatment modality for all risk groups of prostatic adenocaercinoma. Patients are classically stratified according to their Biopsy Gleason score, serum prostate-specific antigen (PSA) level, and clinical stage and are defined as low-risk, intermediate risk or high-risk. A number of studies have confirmed the utility of dose escalation in prostate cancer by improving the local control, freedom from biochemical failure, and freedom from distant metastases. However, conventional fractionation schedules do not permit further escalation beyond doses currently used because of unacceptably high rates of acute and late toxicities using 3D-conformal technologies. Recently, radiotherapy has witnessed the introduction of major technological advances, which have permitted the implementation of intensity modulated radiotherapy (IMRT). IMRT is a further advancement in 3D-conformal radiotherapy. Its primary advantage compared to conventional 3D-conformal treatment techniques, is the ability to produce very sharp dose gradients and to deliver highly conformal target doses with better sparing of normal structures. The benefits of IMRT delivery are particularly pronounced in the context of concave-shaped target-critical structure geometries and, in the treatment of localized prostate cancer, its implementation has resulted in improved toxicity profiles using conventional fractionation regiments.

Extreme hypofractionated image-guided radiotherapy, sometimes referred to as stereotactic body radiotherapy (SBRT) or stereotactic ablative radiotherapy (SABR), aims to deliver even fewer high doses of radiation to the target volume with extreme accuracy and conformity. Growing radiobiological evidence indicates that prostate cancer may have a great sensitivity to large dose per fraction compared to the surrounding normal tissues due to its generally very low alpha/beta ratio, generally believed to be as low as 1.5. Therefore, a potential increase in the therapeutic ratio may be achieved through extreme hypofractionation where the biologically effective dose (BED) to the target tissues is enhanced, while a reduction in the risk of radiation-induced complications is also realized.

While motion tracking devices have been proposed for use in radiotherapy treatments, these motion tracking devices suffer from a number of deficiencies including, but not limited to, being implanted into or proximate to the target tissue. The motion tracking devices thus remain in the patient after the procedure is completed. As described below, this limits the type of post-operative checkups (e.g., imaging) that can be performed and/or the accuracy of such checkups.

There is therefore a need and desire to provide a system that ensures anatomical reproducibility during the medical procedure (especially those in the pelvic region) and provide a less invasive, non-permanent motion tracking device that can be used to detect any movement in tissue which is being treated, such as tissue being subjected to radiotherapy.

### SUMMARY

According to one embodiment, a system to produce anatomical reproducibility during the performance of a medical procedure (treatment) includes a motion tracking device (system) for use in a medical procedure (e.g., radiotherapy) to detect motion of target tissue. The system includes a catheter having a body with a proximal end and an opposite distal end. The catheter body has a first lumen formed therein. The motion tracking device includes a motion tracking assembly that is removably disposed within the first lumen. The motion tracking assembly has a plurality of motion tracking elements that are connected to one another with a connector. The motion tracking elements are spaced apart at fixed distances relative to one another. The motion tracking elements are configured such that movement of any one of the motion tracking elements is detected by the motion tracking device, thereby indicating that the target tissue has moved during the medical procedure. Detecting such movement in real-time allows the operator (e.g., radiation oncologist) to take any necessary immediate remedial action including stopping the medical procedure.

Exemplary applications in which the motion tracking device can be used include but are not limited to: treatment of pelvic malignancies; extreme hypofractionated image-guided radiotherapy for prostate cancer; radiotherapy for cervical cancer; pre-operative and exclusive rectum cancer radiation therapy, etc.

### BRIEF DESCRIPTION OF DRAWING FIGURES

FIG. 1 is a schematic of the bladder anatomy and prostate gland;

FIG. 2 is another schematic of the bladder anatomy and prostate gland;

FIG. 3 is a side elevation view of an exemplary catheter;

FIG. 4A is cross-sectional view of a distal end portion of the catheter showing a motion tracking assembly;

FIG. 4B is cross-sectional view of a distal end portion of another catheter showing a motion tracking assembly;

FIG. 5A is a first transverse cross-section of the catheter showing a connector in a lumen;

FIG. 5B is a second transverse cross-section of the catheter showing one motion detecting element in the lumen;

FIG. 6A is side elevation view of a motion tracking assembly according to one embodiment and removed from the catheter;

FIG. 6B is side elevation view of a motion tracking assembly according to another embodiment and removed from the catheter;

FIG. 6C is side elevation view of a motion tracking assembly according to another embodiment and removed from the catheter;

FIG. 7 is a schematic showing a motion tracking system;

FIG. 8 is a schematic showing insertion of the catheter into the urethra as part of a prostate radiotherapy procedure;

FIG. 9 is a schematic showing insertion of the catheter of FIG. 4A into the urethra and insertion of a rectal balloon as part of a prostate radiotherapy procedure;

FIG. 10 is a schematic showing insertion of the catheter of FIG. 4B into the vagina and cervical canal and insertion of a rectal balloon as part of a cervical radiotherapy procedure; and

FIG. 11 is a side elevation view of a stand that carries a clamp for securing two catheters to one another.

### DETAILED DESCRIPTION OF DRAWING FIGURES

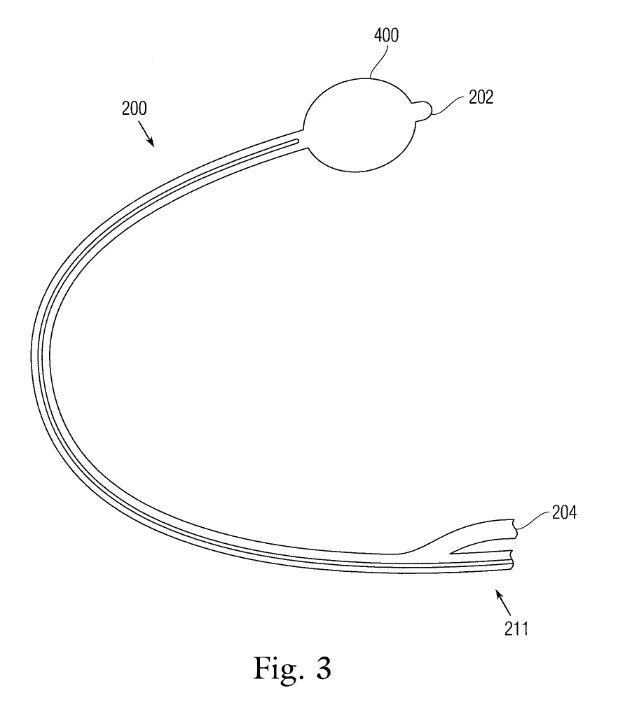

As shown in FIGS. 3-7, the present invention is directed to a system to produce anatomical reproducibility during the performance of a medical procedure and to detect movement of target tissue that is being treated. The system includes a motion tracking device (system) 100 that is used in the medical procedure, such as non-invasive procedures, such as ablative high-dose radiotherapy. By producing anatomical reproducibility and detecting tissue movement, the medical treatment is performed with such increased precision that every healthy organ that may otherwise be at risk is spared from the potential toxicity or ill effects of the medical treatment. As described below, the overall system described and illustrated herein has particular utility for treatment of conditions in the pelvic region of the patient (e.g., pelvic malignancies).

As described below, the motion tracking device 100 provides a non-implantable solution to tracking in real-time any movement of target tissue (e.g., an organ) during the entire medical procedure. In the case in which the medical procedure involves radiotherapy and delivery of a dose of radiation to a specific target, the device 100 allows for increased radiation levels to be used and minimizes the amount of toxicity (acute toxicity) that surrounding tissue is exposed to during the procedure based on the ability to detect the slightest (e.g., 1 mm) movement of the target tissue.

In accordance with one exemplary embodiment, the device 100 is for use in image-guided radiotherapy and more particularly, the device 100 has particular utility in the treatment of pelvic malignancies including extreme hypofractionated image-guided radiotherapy for prostate cancer and treatment of cervical cancer. However and as described herein, the device 100 and/or components thereof can be used in other applications, including other medical procedures, including the treatment of cervical cancer; pre-operative and exclusive rectal cancer radiation therapy, or other procedures in which it is critical to detect any movement of the tissue (organ) during the procedure, etc.

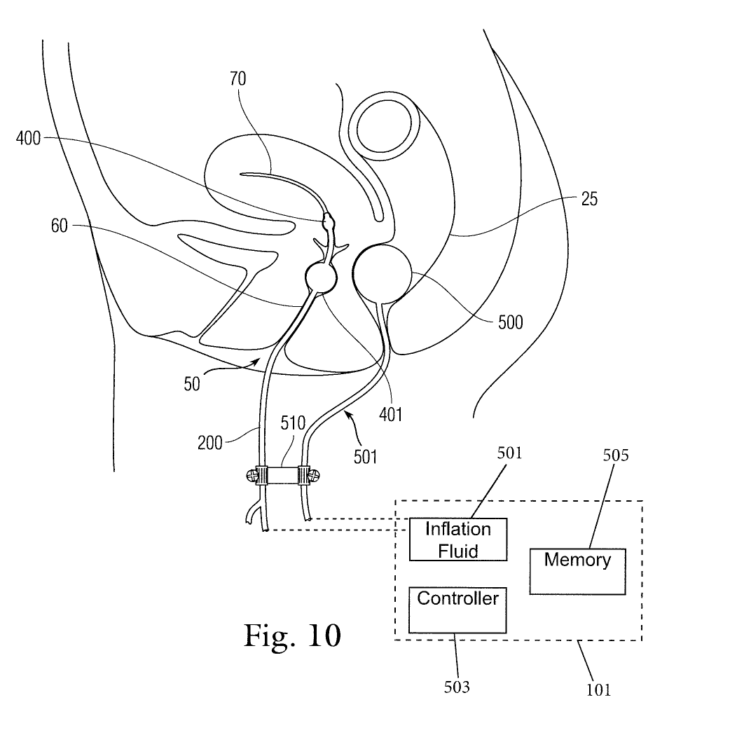

The device 100 is typically one component of a larger overall apparatus (equipment) that is configured to perform multiple operations to effectuate the treatment of the patient and to produce the anatomical reproducibility. This larger apparatus can include a number of operative components including, but not limited to, a means (device) for treating the target tissue (such as delivering radiation in the case of radiotherapy) and imaging means for monitoring the target tissue site. A patient positioning table on which the patient rests during the medical procedure is also typically provided. The overall medical apparatus is typically programmable and is a computer based system and therefore includes a computer that executes software and has a processor (main controller 203) and memory (See FIG. 10).

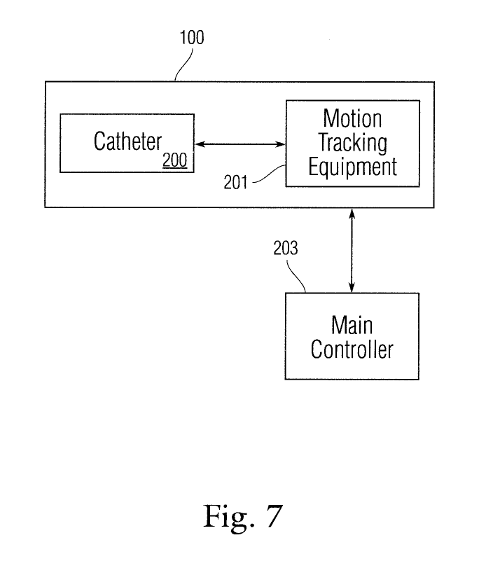

In accordance with the present invention, the motion tracking means (device 100) for detecting any movement of the target tissue during the entire procedure includes a catheter 200 that is configured to provide real-time motion tracking and also includes complementary motion tracking equipment 201 located external to the patient for monitoring the catheter 200 and detecting any movement thereof at the treatment site (See, FIG. 7). As shown in FIG. 7, the catheter 200 is in communication with equipment 201 which in turn is in communication with the main controller 203 such that information concerning any movement of the catheter 200 at the treatment site (and the movement of the target tissue) is delivered to the main controller 203 which processes such information and can initiate any remedial action that is needed.

The catheter 200 is formed of a material that has some flexibility to allow it to navigate the human anatomy. For example, the catheter 200 can be formed of a rubber material, such as a latex rubber, or a silicone material.

The catheter 200 is an elongated structure having a distal end 202 and a proximal end 204. A handle 211 can be located at the proximal end 204. The catheter 200 can be of a steerable type in that it includes components and a mechanism that permits a distal end portion including the distal end 202 to be steerable. Any number of different types of conventional steering mechanisms, including the use of pull wires, etc., can be used for controlling the steering of the distal end 202. The catheter 200 is thus formed of a flexible material (e.g. latex rubber) that allows the catheter 200 to bend when the steering mechanism is operated.

The catheter 200 has one or more lumens formed therein and preferably includes at least two lumens. At least one lumen 210 is formed in the catheter 200 for delivering and positioning a motion tracking assembly 300, which in combination with equipment 201, is designed to track motion, in real-time, of a target structure (e.g., target tissue) to which the motion tracking assembly 300 is associated. The illustrated motion tracking assembly 300 includes elements 302, 304, 306 that can be monitored and tracked in real-time to such a degree that any movement of the elements 302, 304, 306 in any direction can be detected and an alert can be sent to the operator (e.g., radiation oncologist). For example, the motion tracking elements can be members that can be detected using an imaging system (e.g., MRI) and any movement of the elements 302, 304, 306 can be detected using the imaging system. One exemplary element 302, 304, 306 that can be detected and tracked with an imaging system is a radio-opaque element. Alternatively, the elements 302, 304, 306 can be in the form of transponders that send signals to the operator (user) and analysis of the signals allows the operator to detect any movement of any one of the transponders in any direction during the procedure. In each of these systems, the elements 302, 304, 306 are used to establish an initial target position of the treatment site and this initial target position serves as a baseline from which the real-time positions of the elements 302, 304, 306 are compared in order to detect the slightest of movement of one or more of the elements.

Unlike conventional motion tracking elements that are used as guides in medical procedures, such as radiotherapy, the motion tracking elements 302, 304, 306 are not permanently implanted into the target tissue. As discussed above, the permanent implantation of such motion tracking elements prevents certain subsequent imaging of the treated tissue since the elements create artifacts in the images of the treatment site. In other words and in one application, the presence of the motion tracking elements at the treated tissue makes the recorded/captured image (e.g., an MRI image) unreadable. In contrast to these conventional implanted motion tracking elements, the motion tracking elements 302, 304, 306 of the present invention are not permanently implanted into the patient but instead can be freely removed from the patient's body post treatment and therefore, the patient can undergo post-treatment imaging, without any of the above-mentioned disadvantages, to monitor the treated tissue.

The lumen 210 which receives the motion tracking assembly 300 is a longitudinal lumen that extends a length of the catheter 200 and more particularly, extends to a distal end portion of the catheter. In other words, the lumen 210 extends to or proximate to the distal end 204 of the body of the catheter 200.

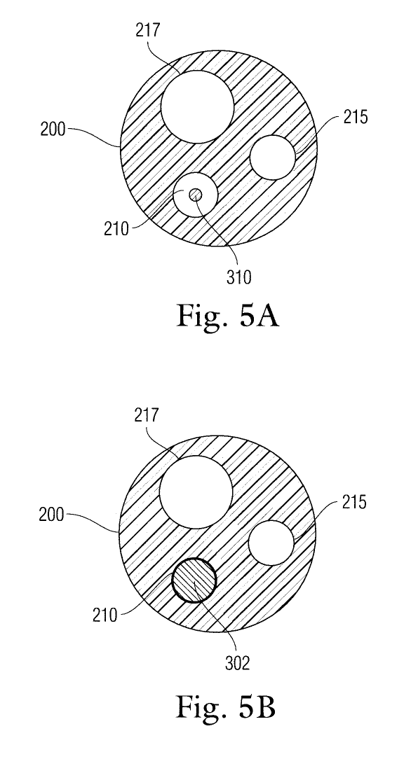

The motion tracking elements 302, 304, 306 of the present invention are contained within the lumen 210 of the catheter 200 which is itself delivered to the target location for serving as an motion tracking device to assist in the delivery of the radiation (in the case of radiotherapy) to a specific target site and in particular, to ensure that the radiation is delivered only to the target site during the entire procedure. In one embodiment, the motion tracking elements 302, 304, 306 are frictionally fit within the lumen 210 so as to secure the elements in place and prevent any movement of the motion tracking assembly 300 within the catheter 200. FIG. 5B shows such friction fit of the elements 302, 304, 306 within the lumen 210. As mentioned herein, the catheter body typically is formed of a rubber material and the elements 302, 304, 306 are formed of either a plastic, glass based material or metal and thus, the rubber of the catheter body grips the outer surface of the elements 302, 304, 306, thereby securing them in place in the lumen 210.

In accordance with the present invention, there is a plurality of motion tracking elements 302, 304, 306 that are fixedly attached to one another at prescribed fixed distances between the individual elements 302, 304, 306. The motion tracking elements 302, 304, 306 are sized and shaped so as to fit within the lumen 210 and are capable of longitudinal movement (e.g. sliding action with frictional contact with the lumen wall) within the lumen 210 to position the motion tracking elements 302, 304, 306 at a desired target location within the lumen 210.

The motion tracking elements 302, 304, 306 can be fixedly connected to one another using a longitudinal connector 310 that connects the motion tracking elements 300 in series in such a way that the relative distances between the motion tracking elements 302, 304, 306 are fixed and thus known. The motion tracking elements 302, 304, 306 in combination with the connector 310 can be thought of as forming the motion tracking assembly 300. Since the catheter 200 is flexible, the longitudinal connector 310 is also flexible so that it can bend and assume a shape similar to the catheter 200 in which it is contained. In the exemplary embodiment, the connector 310 is in the form of a wire, rod, thread, cable, etc., and the motion tracking elements 302, 304, 306 are fixed relative to the connector 310. The connector 310 has sufficient rigidity such that it maintains its longitudinal shape (keeping the elements 302, 304, 306 at the prescribed spaced distances) unless a sufficient force is applied thereto to cause bending thereof.

In one embodiment, the spacing between each of the elements 302, 304, 306 is between about 1 cm to about 2 cm. The shape and size of the elements 302, 304, 306 can also vary depending upon the application (e.g., a diameter of about 4 mm).

Any number of different techniques can be used to fix the motion tracking elements 302, 304, 306 in place along the length of the connector 310 (e.g., wire) and more specifically, the motion tracking elements 302, 304, 306 can be mechanically attached (e.g., using a locker, knots, etc.), can be bonded or adhesively attached, etc. The elements 302, 304, 306 can circumferentially surround the connector 310.

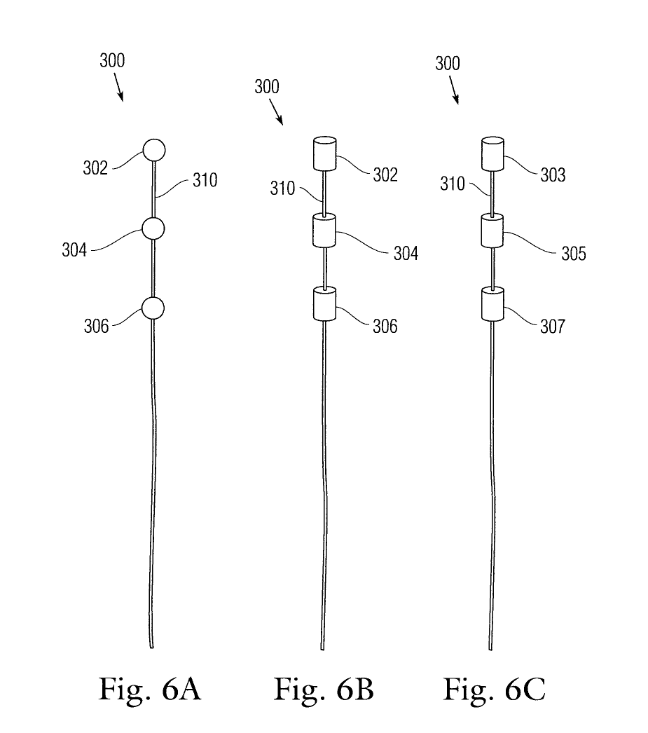

In one embodiment, the motion tracking elements 302, 304, 306 are formed of radio-opaque materials (See, FIG. 6A). As is known, radiopacity is the quality or property of obstructing the passage of radiant energy, such as x-rays, with the representative areas appearing light or white on the exposed film. When radio-opaque markers are used, the equipment 201 is in the form of an imaging device that can detect any movement of any of the markers. Radio-opaque materials include metals and MRI compatible resins. The shape of the radio-opaque markers can vary with the ones in FIG. 6A being spherical in shape.

In another embodiment shown in FIG. 6B, the motion tracking elements 302, 304, 306 are in the form of transponders (e.g., cylindrical shaped) which communicate with an external motion tracking system (localization system—generally shown in FIG. 7 as equipment 201). The communication protocol is of such a type that is safe and can be achieved in the intended applications. More particularly, the transponders can (passively) send unique signals, such as radiofrequency waves, which are detected, analyzed and monitored by the external motion tracking system. The exact location of each transponder can thus be tracked in real-time using this system by analyzing the characteristics of the emitted signals (waves). Each transponder sends a unique signal and thus can be identified. Suitable transponders are commercially available under the trade name Calypso® Surface Beacon® transponders from Varian Medical Systems of Palo Alto, Calif. This type of transponder has an outer glass shell that is held in contact between the lumen wall 210.

FIG. 6C shows a motion tracking assembly that is similar to the transponder assembly of FIG. 6B with the exception that the motion tracking elements 303, 305, 307 do not emit any signals. Instead, these elements 303, 305, 307 are “dummy” transponders that have the same shape and same dimensions as the signal emitting transponders 302, 304, 306 and are inserted into lumen 210 in place of the motion tracking assembly 300 with the signal emitting transponders 302, 304, 306. The use of these transponders 303, 305, 307 is described below.

Since the motion tracking elements 302, 304, 306 provide real-time motion analysis, the operator (e.g., radiation oncologist) can make, quick important decisions about the patient's care including, but not limited to, stopping the radiotherapy when motion of one or more elements 302, 304, 306 falls outside of an acceptable range (e.g., a 1 mm or greater change in position), thereby indicating movement of the tissue that is being treated.

In the illustrated embodiment, there are three motion tracking elements 302, 304, 306 that are connected to one another with connector 310 to form the marker assembly and more specifically, the motion tracking elements 300 include first motion tracking element 302; second motion tracking element 304; and third motion tracking element 306. The first motion tracking element 302 is intended for placement at a first position at the treatment site, the second motion tracking element 304 is intended for placement at a second position at the treatment site and the third motion tracking element 306 is intended for placement at a third position at the treatment site. The first motion tracking element 302 can be thought of as being the distalmost marker, the third motion tracking element 306 as being the proximalmost marker and the second motion tracking element 304 as being the intermediate marker.

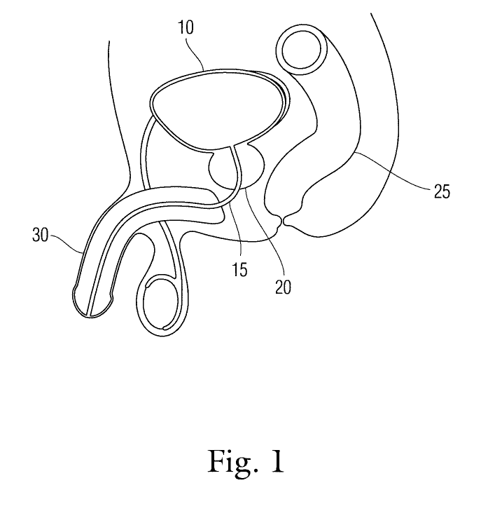

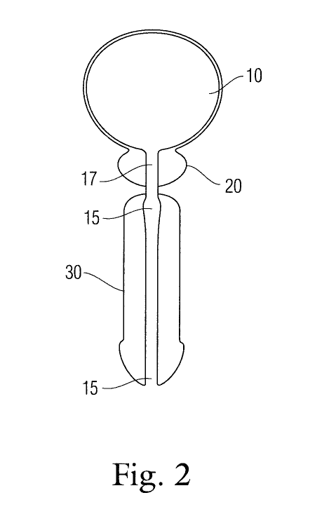

Now referring to FIGS. 1-2 and 8-9, with respect to radiotherapy for the prostate, the catheter 200 is fed through the urethra 15 and the first motion tracking element 302 is positioned at the base of the bladder 10; the third motion tracking element 306 is positioned at the prostatic apex of the prostate 20; and the second motion tracking element 304 is positioned between these two anatomical structures. All three elements lie within the prostatic urethra 17.

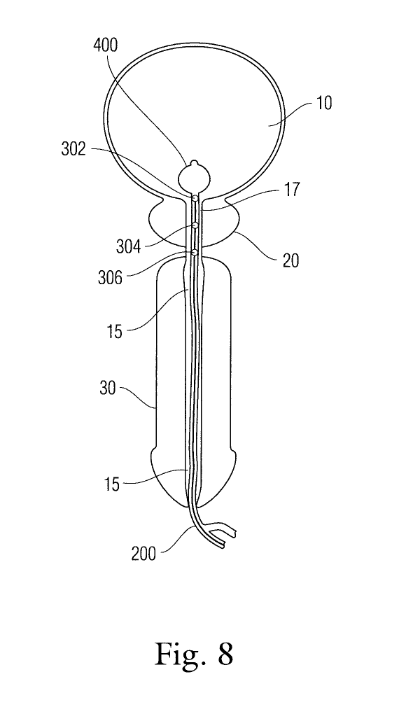

As is known, the prostate 20 is a walnut-sized gland that is located between the bladder 10 and the penis 30. The prostate 20 is just in front of the rectum 25. The urethra 15 runs through the center of the prostate 20, from the bladder 10 to the penis 30, letting urine flow out of the body. The catheter 200 is thus guided within the urethra 15 proximate to these anatomical structures (organs) so as to position the first motion tracking element 302 at the base of the bladder 10 and the third motion tracking element 306 at the prostatic apex (See FIG. 8). In the case of radiotherapy for prostate cancer, it will be appreciated that since the urethra 15 passes through the prostate 20, the positioning of the three motion tracking elements 302, 304, 306 within the prostatic urethra 17 provides motion tracking of the prostate itself without the elements 302, 304, 306 being implanted into the prostate tissue. Further, since the elements 302, 304, 306 are contained within the urethra 15, the motion tracking elements 302, 304, 306 can be withdrawn easily from the patient by being removed through and/or along with the catheter 200 that is contained in the urethra 15.

It will be appreciated that a plurality of motion tracking assemblies can be provided and the operator (e.g., radiation oncologist) then selects the appropriate motion tracking assembly for use based on certain parameters such as anatomical considerations. In particular, the motion tracking assembly is selected which positions the individual motion tracking elements at the proper target positions. In particular, the different motion tracking assemblies 300 have different fixed distances between the individual motion tracking elements (the “marker distances” or “marker spacings”). For example, one motion tracking assembly has first distances between the individual elements 302, 304, 306; another motion tracking assembly has second distances between the individual motion tracking elements; and yet another motion tracking assembly has third distances between the individual motion tracking elements, with first, second and third distances being different from one another. As long as the distances are fixed and known, the distances between the three elements can be different.

For patients with larger prostate glands, the distance between the base of the bladder and the end (margin) of the prostate is greater and therefore, a motion tracking assembly that has greater distances between the individual motion tracking elements is needed in order to position the motion tracking elements at the desired anatomical target points (i.e., organs). The motion tracking assemblies can thus be supplied as a kit that has different marker distances as discussed herein and the operator (e.g., radiation oncologist) can select the motion tracking assembly that has the optimal marker spacings. It will also be appreciated that the motion tracking assemblies can be identified by an identifying name or number, such as a serial number, etc. These identifying features are preferably stored in a database and the software of the system can be configured such that once certain patient specific parameters are inputted, the software can output a recommended (optimal) motion tracking assembly for one specific patient. For example, an image of the internal organs can be used as a basis for measuring the distance between the base of the bladder to the end of the prostate and based on this calculation (measurement), the processor executing the software outputs the specific motion tracking assembly that is recommended for use with one particular patient.

It will also be understood that the motion tracking elements 300 can have any number of different shapes and sizes so long as the motion tracking elements 300 are either clearly visible in the imaging performed as part of a particular application or are otherwise in communication with external equipment 201 that allows the precise position of the motion tracking elements 302, 304, 306 to be determined in real-time. The elements also need to be of a size and shape to be held in place in the lumen 210.

The device 100 is designed to use the motion tracking elements 302, 304, 306 as part of a tracking and locating system (along with equipment 201) that allows any movement of the target tissue to be determined and thus, allows an increased dose of radiation to be delivered due to the increased degree of precision concerning the position of the target tissue during the entire procedure. In particular, motion tracking elements 302, 304, 306 allow for a triangulation calculation to be performed by equipment 201 to determine the coordinates of the catheter 200 throughout the entire procedure. As is known and simply put, triangulation is a way of determining an object's location using the locations of other objects, in this case the location of the motion tracking elements (due to the motion tracking elements being at fixed distances from one another as described herein). Consequently, the movement of the target tissue that is to be irradiated can be detected.

It will also be appreciated that at least in some embodiments, the lumen 210 is a closed ended lumen and the length of the connector 310 (e.g., wire) is known and therefore, the distances from each motion tracking element 300 to a distal end of the connector 310 is also known and the elements do not freely move within the lumen 210. Thus, when the user inserts the connector 310 carrying the motion tracking elements 302, 304, 306 into the lumen, the user feeds the connector 310 through the lumen 210 until the distal end of the connector 310 contacts the closed distal end of the lumen 210. Since the distances of each motion tracking element 302, 304, 306 to the distal end of the connector 310 is known and the distances between the motion tracking elements 302, 304, 306 are fixed, the precise locations of the motion tracking elements 302, 304, 306 within the lumen 210 are known.

However, in other embodiments of the present invention, the precise locations of the motion tracking elements 302, 304, 306 within the lumen 210 are not critical so long as the distances between the motion tracking elements 302, 304, 306 are fixed and are thus known. Since, as described below, the motion tracking elements 302, 304, 306 are positioned relative to organs of the patient, the locations of the motion tracking elements 302, 304, 306 within the lumen 210 are not critical so long as the motion tracking elements 302, 304, 306 do not freely move and change positions within the lumen 210 and the distal end of the catheter 200 is capable of reaching within the patient's body a target position that places the distalmost motion tracking element 302 at a target organ position.

In one exemplary embodiment and as shown in FIG. 3, the catheter 200 is a Foley type catheter that includes the additional lumen 210 for delivering the motion tracking elements 302, 304, 306. As is known, a Foley type catheter is a flexible tube that is often passed through the urethra and into the bladder. The tube typically has two separated channels, or lumens, running down its length. One lumen is open at both ends and allows urine to drain out into a collection bag. The other lumen has a valve on the outside end and connects to a balloon at the tip. The balloon is inflated with sterile water or contrast agent for improved visualization when it lies inside of the bladder in order to stop it from slipping out. Foley catheters are commonly made from silicone rubber or natural rubber or latex. The portion of the Foley catheter that expands distally beyond the balloon includes the urine port which passes the urine into the urine lumen.

The catheter 200 of the present invention preferably includes a means for securing the catheter and the target location and in particular, the catheter 200 can have an inflatable balloon 400 that is formed along the body of the catheter 200. The balloon 400 inflates radially outward relative to the body thereof.

As mentioned above, the present system is designed to produce anatomical reproducibility during the medical procedure (treatment). This involves securely stabilizing the tissue to be treated as well as the components of the system that are delivered to the target tissue site. For example, the balloon 400 can be inflated using any number of conventional inflation techniques/mechanisms including, but not limited to, the use of an inflation fluid that is delivered through the catheter body to the balloon 400. More specifically, the catheter body can include an additional lumen 215 (FIG. 5A/5B) for delivering the inflation fluid to the balloon 400. A controller (such as controller 203), which can be in the handle of the catheter, is used for controlling the inflation of the balloon and more specifically, controls the inflation speed and/or inflation level. This balloon 400 is designed to securely anchor the catheter at the designed location and thus prevent unwanted movement of the catheter 200 during the non-invasive procedure, such as ablative high-dose radiotherapy. It will be appreciated that the balloon 400 does not adversely impact the use and visibility of the motion tracking elements during the imaging process.

As described herein, the catheter 200 and related equipment can be part of a computer implemented system that includes other equipment such as imaging equipment. All of the equipment can communicate with a central processor or the like to control the operation of the various pieces of equipment.

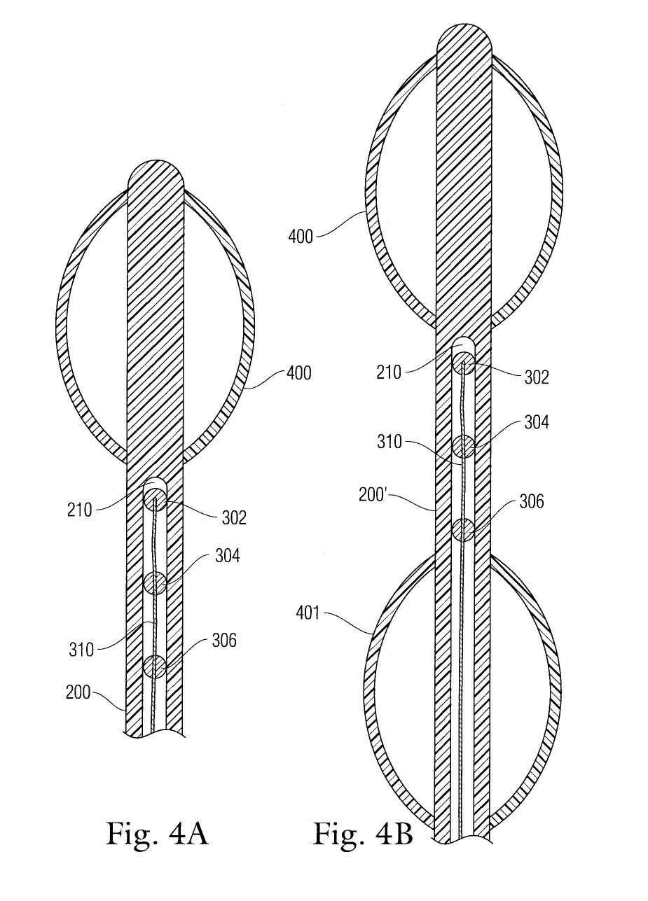

Preferably, the elements 302, 304, 306 are spaced from the balloon 400 (i.e., not contained within the balloon 400). For example, the elements 302, 304, 306 can be located proximal to the balloon 400 as shown in FIG. 4A.

FIG. 4B shows another catheter 200′ which is particularly for use in a cervical treatment, such as during a cervical cancer treatment. The catheter 200′ is very similar to the catheter 200 with the exception that the catheter includes a second balloon 401. The second balloon 401 is located proximal to the motion tracking elements 302, 304, 306, while balloon 400 is located distal thereto. As described below in reference to FIG. 10, this type of catheter 200′ is intended for use in applications (e.g., cervical cancer treatment) where two balloons 400, 401 are helpful to anchor the catheter. It will be appreciated that there are two separate lumens formed in the catheter for independent inflation of the two balloons 400, 401. In other words, one inflation lumen carries inflation fluid for inflating balloon 400, while the other inflation lumen carries inflation fluid for inflating balloon 401. The use of the motion tracking assembly 300 is the same in this catheter as in the catheter 200.

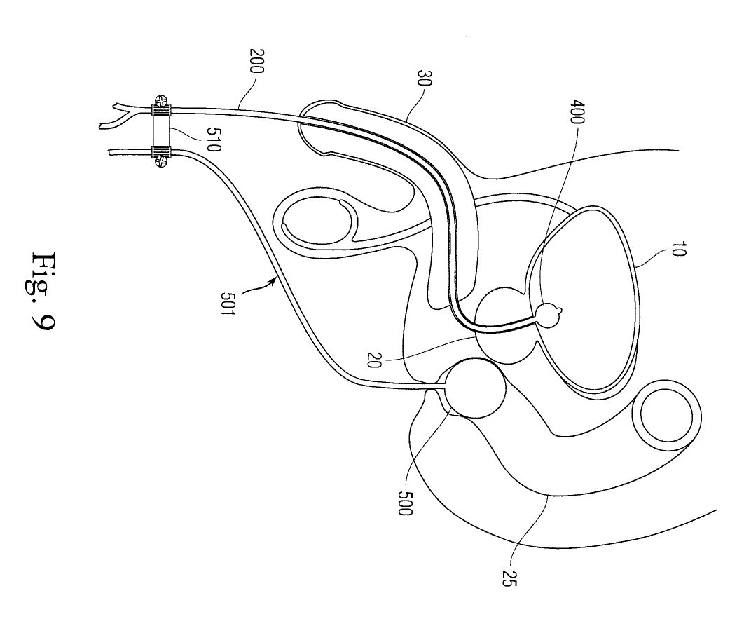

In yet another aspect of the present invention and as shown in FIG. 9, another balloon is used as part of the radiotherapy of the prostate and more specifically, a rectal balloon 500 which is part of an assembly 501 is used. The use of the rectal balloon 500 during radiotherapy significantly reduces prostate motion. Prostate immobilization thus allows a safer and smaller planning target volume margin. It also can spare the anterior rectal wall (by its dosimetric effects) and reduces the rectal volume that receives high-dose radiation (by rectal wall distension). All these factors decrease rectal toxicity achieved by radiotherapy, despite dose escalation and higher-than-conventional fraction size.

In accordance with one embodiment of the present invention, the device 100 of the present invention is configured to mate with the rectal assembly 501. More particularly, the catheter 200 can be configured to mate with the balloon 500, thereby providing immobilization of the prostate and further securement of the catheter 200. Any number of different mechanisms can be used to couple the catheter 200 to the rectal assembly 501. For example, a mechanical attachment, magnetic attachment, or releasable bond can be achieved between the two. Such connection between the rectal balloon assembly 501 and the catheter 200 is at a location external to the patient. For example, as shown in FIG. 9, a clamp type mechanism 510 can be used to clamp the proximal portion of the catheter 200 to the proximal portion of the rectal balloon assembly 501, thereby locking the two structures to one another. This is yet another technique for ensuring that the positions of the instruments remain fixed to one another which assist in determining any movement of the target tissue during the entire procedure.

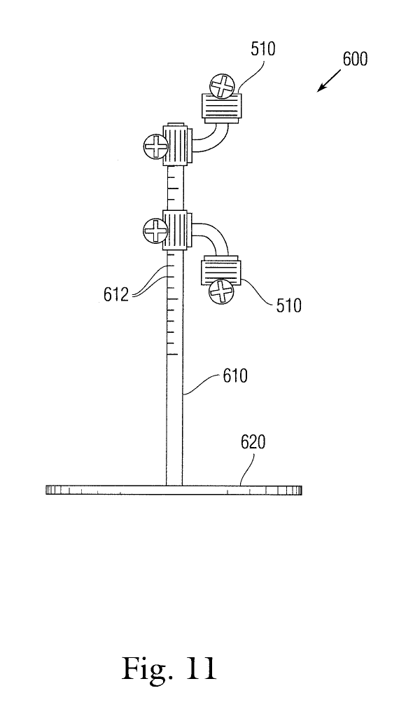

As shown in FIG. 11, it will also be appreciated that the clamp 510 can be part of a stand 600 (pedestal) that permits the clamp 510 to be moved along a support member 610 (vertical support) into a desired position to accommodate an individual patient. The support member 610 is connected to a base member 620. The support member 610 can include index markings 612 that allow the clamp 510 to be reproducibly placed into one defined position along the vertical support 610. In one design, the clamp 510 can also be adjusted in a lateral direction (telescopic) to provide increased clamping versatility between the two structures 200, 501.

In the case of a steerable catheter, an additional lumen can be used to house the components used for steering the catheter 200 and in particular, steering wires (pull wires) can be routed within one lumen that is formed internally within the body of the catheter 200. Such lumen is similar to the other lumens in that it is a longitudinal lumen that extends the length of the catheter 200. Additional lumens, such as lumen 217 for removal of urine 217 can be formed in the catheter body.

As mentioned herein, the motion tracking device 100, including catheter 200, can be used in any number of different medical procedures, including radiotherapy applications. The below examples are for illustrative purposes only and are not limiting of the scope of the present invention.

### Example—Prostate Radiotherapy

As previously mentioned, in one exemplary embodiment, the motion tracking device 100 and in particular, the catheter 200 thereof is used as part of a radiotherapy treatment for prostate cancer. Referring to FIGS. 8-9, the first step is to insert the catheter 200 into the urethra 15 with the motion tracking assembly 300 being inserted into the dedicated channel (lumen 210) of the catheter 200. The catheter 200 is positioned within the urethra 15 until the motion tracking elements 302, 304, 306 are in desired positions within the prostatic urethra 17. As mentioned hereinbefore, the distalmost motion tracking element 302 is positioned at the base of the bladder and the proximalmost motion tracking element 306 being positioned at the prostatic apex. Thus, all three motion tracking elements 302, 304, 306 are located in the region of the urethra 15 that passes through the prostate 20. The precise locations of the motion tracking elements 302, 304, 306 can be observed in real-time using imaging and/or tracking equipment 201. Once the motion tracking elements 302, 304, 306 are in the desired locations relative to the surrounding organs, the catheter 200 can be secured in place within the urethra 15 by inflating the balloon 400 (FIG. 3) that is associated with the catheter 200. The inflation of the balloon 400 to a prescribed inflation level results in the catheter 200 being securely positioned within the surrounding vessel, in this case, the urethra 15.

The rectal balloon 500 is inserted into the rectum 25 and is inflated to a prescribed inflation level that while still comfortable for the patient results in the rectum 25 applying pressure on the prostate 20 and maintains the prostate 20 in a stabilized position.

Once the catheter 200 is secured within the prostatic urethra 17 and the rectal balloon 500 is inflated to stabilize the prostate 20, thereby maintaining the prostate in a fixed position, imaging is performed to determine the precise location (coordinates) of each of the motion tracking elements. These coordinates of the motion tracking elements serve as a baseline from which any subsequent movement of the motion tracking elements is determined and allows an alert in real-time to be sent to the operator in the event that if at any time during the procedure, movement of one or more of the motion tracking elements 302, 304, 306 beyond a permitted range (range of tolerance) is detected.

The non-invasive procedure, such as ablative high-dose radiotherapy, is initiated and during the entire procedure, the positions of the motion tracking elements 302, 304, 306 are monitored in real-time. It is common for the prostate to shift slightly (e.g., 5 mm or more) within about 3 minutes after initiating the treatment. As mentioned herein, the manner of monitoring depends on the type of elements 302, 304, 306 being used in that either direct imaging of the elements 302, 304, 306 can be performed or monitoring of emitted signals (e.g., radiofrequency waves) can be performed.

An alert can be generated at any point in time when one or more of the motion tracking elements 302, 304, 306 moves beyond a permitted degree of motion (e.g., a movement of more than 1 mm in any degree of motion). The alert can be an audio and/or visual alert. Alternatively, the system can have an automatic shut off feature in that the treatment is automatically stopped (e.g., auto power shut off) when movement outside the permitted range occurs.

Once the medical procedure has been completed, the rectal balloon 500 is deflated and the catheter 200 is removed by deflating the balloon 400 and then withdrawing the catheter 200 from the urethra 15.

Subsequent post-operative monitoring of the treated tissue can easily be performed using conventional techniques including post-operative imaging (e.g., MRI), since no components of the motion tracking device 100 remain within the patient's body after the medical procedure is completed.

### Example—Prostate Radiotherapy Using Transponders for Motion Tracking

When the elements 302, 304, 306 are in the form of the transponders that emit signals, as described herein, these type of transponders cannot remain in the patient's body when imaging (such as MRI) is performed during the medical treatment. As a result, the motion tracking device of FIGS. 6B and 6C are used interchangeably. More specifically, the motion tracking device containing the elements 303, 305, 307 (FIG. 6C) is initially disposed within the lumen 210 and the catheter 200 is inserted into the urethra 15 as described above. Imaging equipment (MRI) is used to position and record the set positions of the elements 303, 305, 307. The device and elements 303, 305, 307 are removed from the lumen 210 and then the device with the signal emitting elements 302, 304, 306 is inserted into the lumen 210. The medical treatment (e.g., radiotherapy) is then begun.

During the procedure, imaging is performed to observe the progress of the medical treatment; however, before such imaging is performed, the device with the signal emitting elements 302, 304, 306 is removed from the lumen 210 and the device with the “dummy” elements 303, 305, 307 is inserted into the lumen 210 since imaging cannot be performed with elements 302, 304, 306 in place at the target tissue location. It will be appreciated that the device carrying the signal emitting elements 302, 304, 306 and the device with the “dummy” transponder elements 303, 305, 307 can be interchangeably used in the lumen 210 during the procedure.

### Example—Cervical Radiotherapy

As previously mentioned, in one exemplary embodiment, the motion tracking device 100 and in particular, the catheter 200′ (FIG. 4B) thereof is used as part of a radiotherapy treatment for cervical cancer. Referring to FIG. 10, the first step is to insert the catheter 200′ into the vagina 50 with the motion tracking assembly being inserted into the dedicated channel (lumen 210) of the catheter 200′. The catheter 200′ is advanced through the vagina 50 and into the cervical canal 60 toward the uterus 70 until the motion tracking elements 302, 304, 306 are in desired positions within the cervical canal 60. In one application, all three motion tracking elements 302, 304, 306 are located in the cervical canal 60. The precise locations of the motion tracking elements 302, 304, 306 can be observed in real-time using imaging and/or tracking equipment 201. Once the motion tracking elements 302, 304, 306 are in the desired locations relative to the surrounding organs, the catheter 200′ can be secured in place within the cervical canal 60 by inflating the balloon 400 within the cervical canal 60 and inflating the balloon 401 within the vagina 50. The inflation of the balloons 400, 401 to prescribed inflation levels results in the catheter 200′ being securely positioned within the cervical canal 60.

The rectal balloon 500 is inserted into the rectum 25 and is inflated to a prescribed inflation level that while still comfortable for the patient results in the rectum 25 applying pressure on the cervix.

Once the catheter 200′ is secured within the cervical canal 60 and the rectal balloon 500 is inflated to stabilize the cervix, thereby maintaining the cervix in a fixed position, imaging (MRI) is performed to determine the precise location (coordinates) of each of the motion tracking elements 302, 304, 306. These coordinates of the motion tracking elements serve as a baseline from which any subsequent movement of the motion tracking elements is determined and allows an alert in real-time to be sent to the operator in the event that if at any time during the procedure, movement of one or more of the motion tracking elements 302, 304, 306 beyond a permitted range (range of tolerance).

The non-invasive procedure, such as ablative high-dose radiotherapy, is initiated and during the entire procedure, the positions of the motion tracking elements 302, 304, 306 are monitored in real-time. As mentioned herein, the manner of monitoring depends on the type of elements 302, 304, 306 being used in that either direct imaging of the elements 302, 304, 306 can be performed or monitoring of emitted signals (e.g., radiofrequency waves) can be performed.

An alert can be generated at any point in time when one or more of the motion tracking elements 302, 304, 306 moves beyond a permitted degree of motion (e.g., a movement of more than 1 mm in any degree of motion). The alert can be an audio and/or visual alert. Alternatively, the system can have an automatic shut off feature in that the treatment is automatically stopped (e.g., auto power shut off) when movement outside the permitted range occurs.

Once the medical procedure has been completed, the rectal balloon 500 is deflated and the catheter 200′ is removed by deflating the balloons 400, 401 and then withdrawing the catheter 200′ from the vagina 50.

It will therefore be appreciated that the combination of the rectal balloon 500 and the catheter 200 (or any of the catheters disclosed herein) can be used in a number of different applications including prostrate and cervix treatments as disclosed herein.

### Additional Examples

As shown in FIG. 10, the rectal balloon 500 can be part of a catheter 501 that includes a lumen that is operatively connected to a source of inflation fluid 502 and similarly, the catheter 200 is operatively connected to a source of inflation fluid for controllably inflating the one or more balloons that are associated with the catheter 200. It will be understood that a single source of inflation fluid 502 can be used to inflate the balloons associated with the rectal balloon and the one or more balloons associated with the catheter 200 or multiple source of inflation fluid 502 can be used. FIG. 10 also shows that a controller 503 can be operatively connected to the source of inflation fluid 502 to control the flow of the inflation fluid to the balloons associated with the rectal catheter and the second catheter 200. The system can thus be part of a computer implemented system 101 with the controller 503 being operatively connected to a main controller (processor) that is part of a main controller/processor, such as a PCB. The system can include a computing device, such as a desktop, laptop, tablet, etc., which can include an input device for inputting data and also a display on which information can be displayed. The computing device can be in communication with other systems/devices used in the treatment, such as imaging equipment and radiotherapy equipment. The computer implemented system thus can execute software to control operation of the various components of the system and record and store data, such as patient images and measurements obtained by the various instruments and components of the system. Conventional communications protocol can be used to allow communication between the various components.

The present system thus can be operated to controllably inflate each balloon that is part of the system and then once each balloon has reached a prescribed optimal inflation level, the system can store in memory 505 such optimal threshold inflation levels for the various balloons along with other identification information, such as patient information and/or catheter identification information, such as a unique identifier (e.g., serial number) for each catheter. In this way, the exact equipment for a patient can be reused for later treatments and later procedures, such as imaging procedures, and the stored data/measurements, such as inflation levels, allow the states of the equipment to be reproduced.

As will be described herein, for each patient, a set of catheters can be selected for use in the treatment of this particular patient. The patient specific identification information thus can include a unique identifier for the specific patent and each of the catheters includes a unique identifier that is associated with the unique identifier for the specific patent. The catheters can be used in an initial fitting in which the optimal inflation levels are determined for this specific patient and then used in subsequent applications including both subsequent treatment and imaging applications as discussed herein. By first determining and then storing the inflation level details for each patient, anatomical reproducibility can be achieved and is an important advantage offered by the present system. Anatomical reproducibility is important since the procedures described herein utilize a plurality of catheters that are inserted and removed from the patient's body numerous times over the course of the treatment and thus, it is desired that when the catheters are subsequently reinserted into the body and the respective balloons are inflated, this is done to produce the same anatomical conditions (stabilization of the target tissue, etc.) as was recorded in the initial procedure (i.e., the baseline procedure).

According to one embodiment, the following is an exemplary procedure to achieve optimal anatomical reproducibility in prostate high-precision radiotherapy. The need for safe dose escalation in prostate cancer radiotherapy demands that accurate delineation of the target organ and adjacent critical structures is performed. MRI provides optimal resolution of pelvic organs and has become a necessary tool for target delineation. Due to the well-known mobility of pelvic organs, large volumes of healthy non-target tissues have traditionally been exposed to high radiation doses contributing to the relatively high rates of post-irradiation normal tissue complications. To improve quality of prostate irradiation and safely dose escalate to increase cure rates treatment planning and delivery, the system and method of the present invention incorporates the following features:
1) accurate anatomic delineation of pelvic organs and their reproducibility via motion mitigation;
2) high level of anatomical reproducibility to assure precise dose deposition as per treatment plan with high dose gradients between the target and surrounding organs at risk;

a) Verification of occurrence of said reproducibility and stability during treatment delivery.

Assurance of this may be achieved via on-line tracking using beacon transponder technology once image guidance has confirmed accurate set-up within tolerance limits. Treatment may be interrupted should motion occur beyond set safety threshold (e.g., beyond 2 mm which is one exemplary threshold value—however, it will be understood that other threshold values can be inputted by the user based on certain considerations, such as patient anatomy).

### Procedures to Achieve Anatomical Reproducibility and Stability & Set-Up for Tracking Procedures

A) During the simulation procedure necessary for treatment planning the following exemplary steps can be taken to create the conditions for a reproducible pelvic anatomy:
   1) an enema to clean the large bowel is performed within 20 minutes (or other selected time) of the procedure;
   2) the patient voids the bladder immediately prior to the procedure;
   3) the patient is placed on the CT scanner supine;
   4) a Foley catheter preloaded with a plurality, such as three, beacon transponders (or radiopaque markers) with a predetermined spacing based on the known volume of the patients prostate (minimum spacing between center to center is about 1 cm);
   5) with the patient lying to the side an endorectal balloon with an indexed shaft is inserted to a depth determined by size of prostate;
   6) an initial volume of about 100 cc of air is placed inside endorectal balloon;
   7) a CT scan is acquired to check accuracy of balloon position (i.e. alignment with skeletal anatomy, no bending);
   8) Balloon filling should be such that no pockets of air are detected around it, so as to distort the extension of the rectal wall in an unpredictable and non-reproducible fashion;
   9) Optimal balloon filling is determined based on the patient's specific anatomy: the rectal wall should be distended beyond physiological conditions (i.e. higher than the volume of the patient's rectal ampulla) so that no pockets of gas may occur (as a result, the selected balloon inflation level is selected to result in such desired distention of the rectal wall). Additionally, in patients with low-lying small bowel loops higher volumes may be needed to push these out of the treatment field. CT scans with incremental volumes are acquired until one with the optimal volume is obtained;
   10) Personalized balloon fillings typically range between about 100 and about 200 cc. No significant patient discomfort has been observed up to 150 cc and compliance has been very good when higher volumes (up to 200 cc) were deemed necessary.
   11) Balloon volumes of about >100 cc have been shown to produce significant motion mitigation by virtue of the pressure exerted on the prostate which is effectively locked between the balloon itself and the pubic bones. It will be appreciated that the ideal (optimal) balloon volume is dependent on the patient's pelvic anatomy and size of the gland.
   12) At the end of the CT simulation, the loaded Foley catheter is replaced with a MRI compatible one (see below) and the patient is moved in for MRI scanning with the optimal balloon volume and void bladder.
B) Creating the ideal (optimum) conditions for online tracking with beacon transponders.
   1) Shortcomings of the implanted beacon transponders:
      Invasiveness. Transperineal implant requires local anesthesia.
      Image artifacts on MRI scans; these are the most significant limiting factor for the use of permanently implanted beacon transponders as they produce null signals with radii approximately 1.5 cm and length approximately 4 cm, effectively precluding the use of MRI treatment-planning (if the scan is performed post-implant) and in post-treatment assessment.
   2) Modification of the procedure to eliminate MRI artifacts induced by the transponders:
      for MRI to be flawlessly included in the treatment-planning and post-treatment assessment workflow, a novel approach to obviate the need of permanent implant has been devised using a catheter-based technique.
      The beacon transponders are placed within the prostate gland via transurethral insertion of a pre-loaded catheter and are removed at the end of the CT simulation procedure to allow for MRI image acquisition using a regular equivalent size Foley catheter which can be loaded with dummy (MRI compatible) markers.
      MRI scans in the predefined anatomical geometry with appropriately inflated balloon can, therefore, be acquired in treatment position to accurately contour the target volume.
      Accurate image fusion between CT scan acquired with ideal balloon filling for specific patient anatomy and MRI scan with identical conditions can be performed with appropriate image fusion software.
      The location of the urethra is clearly identified on the fused CT/MRI set by virtue of the catheter. With an about 2 mm expansion around the catheter (in one embodiment), accurate negative dose-painting to cool off the urethra and reduce the risk of treatment-related GU toxicity is successfully achieved.
      The beacon transponders-loaded catheter is reinserted only prior to treatment delivery to exploit the accuracy in patient set-up and tracking capabilities afforded by the tracking device.
      Post-treatment MRI are, therefore, not precluded as no beacon transponders (or other markers) are left intra-prostatically at the end of the treatment.

### Treatment Delivery: Anatomical Reproducibility and Tracking

1) To assure accurate anatomical reproducibility, the patient undergoes the same preparation procedures as per simulation (enema, bladder voiding).
2) The nurse inserts the loaded catheter and endorectal balloon and fills them as per the volumes established during simulation and used for treatment planning (i.e., the stored inflation levels).
3) When beacon transponders are used, the system will already identify set-up accuracy within about +/−1 mm.
4) A confirmatory cone-beam CT (CBCT) can be used as reference is acquired and physician determines ID anatomical reproducibility is acceptable (i.e., within approximately 1 mm) with respect to all pelvic organs compared to planning CT/MRI fusion.
5) Minor adjustments may be needed in balloon position to achieve optimal anatomical reproducibility as seen by matching CBCT and planning CT scans. A 6 degree of freedom (6DoF) couch is used in one exemplary practice to correct subtle discrepancies.
6) CBCT may be reacquired until matching is deemed optimal.
7) Delivery starts with beacon transponders online signal closely monitored to assure motion is within the preset value (i.e., about 2 mm margins) (typically ˜1 mm in all directions).
8) Treatment may be temporarily interrupted if the 2 mm threshold is exceeded (<5% of cases based on current experience).
9) Treatment is completed in 5 sessions or other amount based on patient need. A confirmatory CBCT is acquired at the end of each session to prove accuracy of anatomical and reproducibility and stability by imaging. A full report of target motion is produced by the online tracking system.

The present invention is thus directed to a system that produces anatomical reproducibility during a medical treatment and includes a motion tracking device (tool) that can be used in an apparatus that is configured to perform a medical treatment, such as a non-invasive procedure, such as ablative high-dose radiotherapy, etc. If excessive movement (e.g., movement outside of an acceptable range) is detected by the motion tracking device, then an alert can be sent and the radiotherapy can be automatically stopped. For example, the main controller can send a signal to the radiotherapy instrument that applies the radiation to cause said radiation to stop.

One of the primary advantages of the present system is that healthy tissue (organs) that is at risk during the medical treatment due to its close proximity to the treated tissue (i.e., irradiated tissue) is spared. In the case of prostate radiotherapy, the urethra and surrounding structures, such as neurovascular bundles, are spared; in the case of rectal cancer radiation therapy, the sphincter is spared, etc. These are advantages not possible with conventional equipment and methods.

The motion tracking device is inserted into a vessel or other body part with a portion of the motion tracking device being disposed outside of the body. The positions of trackable motion tracking elements of the device are registered and provide a baseline from which subsequent measurements are based. Unlike traditional motion tracking methods, the device, including the motion tracking elements, of the present invention is completely removable from the treatment site in that the device can be withdrawn from the patient's body once the procedure is completed. This removal offers a number of advantages over the prior art devices and methods in that subsequent post-operative imaging (e.g., MRI) can be performed without any artifacts being caused by the motion tracking elements since they are not permanently implanted at the treatment site. This permits necessary imaging during the medical procedure to be performed and also post-operative checkups to be performed and allows more reliable imaging of the target tissue to be performed.

Broadly speaking, the catheter 200 can thus deliver motion tracking elements (that are part of a motion tracking device) into a patient's body in a non-implantable, removable manner. The catheter 200 can thus be a tool that can be used in any number of different medical procedures to monitor the progress of the medical treatment and permit more aggressive medical procedures to be implemented, while not subjecting healthy tissue in the surrounding areas to toxicity and/or other ill effects of the medical procedure. In other words, the present system spares the healthy tissue due to both the anatomical reproducibility and the motion tracking capability of the present system.

It will also be appreciated that the motion tracking assembly can include means for anchoring the assembly within the lumen 210. For example, a coupling member or the like that is associated with the motion tracking assembly (e.g., associated with the connector 310) can attach to a complementary coupling member that is part of the catheter to releasably secure the connector 310 to the catheter 200.

As discussed herein, the present system provides for optimal anatomical reproducibility as well as motion tracking to ensure safety is achieved during high-precision radiotherapy. Both of these components allow for such increased radiation doses that are described herein.

1. A system for providing anatomical reproducibility and detecting motion of either prostate target tissue or cervix target tissue being treated by radiotherapy comprising:
a first catheter having a body with a proximal end and an opposite distal end, the catheter body having a first lumen formed therein, wherein the first catheter includes:
a motion tracking device that is removably disposed within the first lumen, the motion tracking device having a plurality of motion tracking elements that are connected to one another with a connector, the motion tracking elements being spaced apart at fixed distances relative to one another;
a first inflatable balloon for securely positioning the first catheter at a target location, wherein the first inflatable balloon is configured to be inflated to different inflation levels depending upon patient anatomical considerations;
a second catheter having a second inflatable balloon for insertion into a rectum of the patient, the second inflatable balloon being configured to be inflated to different inflation levels depending upon patient anatomical considerations;
at least one controller operatively coupled to the first inflatable balloon and the second inflatable balloon to permit the first inflatable balloon to be inflated to a first inflation level and the second inflatable balloon to be inflated to a second inflation level, wherein the second inflation level is selected to cause distention of the rectal wall to such a degree that either the prostate target tissue or the cervix target tissue is stabilized due to a force applied by the distended rectal wall to the prostate target tissue or the cervix target tissue; and
memory for storing patient specific identification information and the first and second inflation levels, wherein the memory is accessible by the controller;
wherein the motion tracking device is configured to detect motion of the target tissue and when the motion tracking device detects that either the prostate target tissue or the cervix target tissue moves a distance that is greater than a predetermined value, the radiotherapy is stopped.

2. The system of claim 1, wherein the plurality of motion tracking elements comprises three motion tracking elements.

3. The system of claim 1, wherein the motion tracking elements comprise radio-opaque markers that are fixed to the connector.

4. The system of claim 1, wherein the motion tracking elements comprise transponders that emit unique signals that can be detected external to the patient's body.

5. The system of claim 1, wherein the connector comprises an elongated structure to which the motion tracking elements are fixed.

6. The system of claim 5, wherein the elongated structure comprises one of a wire and cable.

7. The system of claim 1, wherein the catheter body is flexible and the connector is flexible to accommodate bending of the catheter body.

8. The system of claim 1, wherein the first lumen comprises a longitudinal channel formed along a length of the catheter body and the connector comprises an elongated structure that carries the motion tracking elements, the connector being freely insertable and removable from the first lumen.

9. The system of claim 8, wherein the connector includes a means for securing the connector within the first lumen at a set position.

10. The system of claim 1, wherein the plurality of motion tracking elements comprise three motion tracking elements that are spaced apart such that a distalmost motion tracking element can be positioned at a base of a bladder and a proximalmost motion tracking element can be positioned at a prostatic apex.

11. The system of claim 1, wherein the connector passes through the motion tracking elements.

12. The system of claim 1, further including a mechanism for fixedly connecting the first catheter to the second catheter at a location external to the patient.

13. The system of claim 12, wherein the mechanism comprises a clamp mechanism that includes a first clamp portion that clamps to the first catheter and a second clamp portion that clamps to the second catheter and prevents relative movement between the first and second catheters.

14. The system of claim 1, wherein the patient specific identification information includes a unique identifier for the specific patent and each of the first and second catheters includes a unique identifier that is associated with the unique identifier for the specific patent.

15. The system of claim 1, wherein the predetermined value comprises a distance greater than about 2 mm.

16. The system of claim 1, wherein the second balloon is inflated with between about 100 cc and about 200 cc of air.

17. The system of claim 1, wherein the second inflation level is a level at which no pockets of air are present around the second balloon.

18. The system of claim 1, further including at least one imaging device for imaging the patient to detect at least a location of the first and second balloons.

19. A method for producing anatomical reproducibility and for tracking motion of a target tissue that is to be treated during a medical procedure comprising the steps of:
delivering a first catheter to a location at or proximate to the target tissue within a body of a patient, the catheter including a lumen in which a motion tracking device is disposed, the motion tracking device being configured to detect movement of the catheter during the medical procedure;
inflating a first balloon that is associated with the first catheter to a first inflation level that results in the first catheter being anchored relative to the target tissue;
delivering a second catheter to a rectum of the patient and initially inflating a second balloon that is associated with the second catheter and located within the rectum to an initial inflation level;
determining a position of at least one of the first and second balloons with imaging equipment;
inflating the second balloon to a target inflation level, the target inflation level being an inflation level which causes distention of a rectal wall to such a degree that either the target tissue is stabilized due to a force applied by the distended rectal wall to the target tissue and no pockets of air are present around the second balloon;
replacing the first catheter with a third catheter that is compatible to imaging of the patient using imaging equipment to allow locations of the first and second catheters to be recorded;
storing in memory the target inflation level of the balloon, the first inflation level of the first balloon, as well as patient specific identification information;
replacing the third catheter with the first catheter that includes the motion tracking device;
inflating the first balloon to the first inflation level and the second balloon to the target inflation level and positioning the first and second catheters to be at located at least substantially at the locations previously recorded; and
initiating radiotherapy of the target tissue while monitoring the motion tracking device to detect if the target tissue moves a distance that is greater than a predetermined value, and if the target tissue moves more than the determined value, the radiotherapy is stopped.

20. The method of claim 19, wherein the motion tracking device comprises a plurality of motion tracking elements that are fixedly attached to a rigid elongated structure that is inserted into the lumen of the first catheter.

21. The method of claim 19, wherein the first catheter is inserted into a vessel formed in the patient's body and advanced until the motion tracking device is at a target position relative to the target tissue.

22. The method of claim 21, wherein the vessel comprises the urethra.

23. The method of claim 19, wherein the motion tracking device includes a plurality of radio-opaque markers disposed within the lumen and the step of detecting movement comprises performing imaging of the target tissue and detecting any change in location coordinates of any one of the markers.

24. The method of claim 19, wherein the target tissue comprises one of prostate tissue and cervix tissue.

25. A method for performing extreme hypofractionated image-guided radiotherapy for treating prostate cancer comprising the steps of:
delivering a first catheter within a body of the patient, wherein the first catheter includes a catheter body having a proximal end and a distal end, the catheter body having a first lumen formed therein, the first catheter having a motion tracking device disposed within the first lumen, the motion tracking device having a plurality of motion tracking elements that are connected to one another with a connector, the motion tracking elements being at fixed distances relative to one another;
positioning a distalmost tracking element of the plurality of motion tracking elements at a base of the bladder of the patient;
positioning a proximalmost tracking element of the plurality of motion tracking elements at a prostatic apex;
stabilizing the prostrate by inserting a second catheter into the rectum and inflating an endorectal balloon such that the rectal wall distends a distance to apply pressure to the prostate, whereby the prostate is stabilized;
recording locations of the first and second catheters and an inflation level of the endorectal balloon;
performing imaging of the target tissue and detecting any change in location coordinates of any one of the motion tracking elements which result in the target tissue moving a distance that is greater than a predetermined value and if the target tissue moves a distance greater than the predetermined value, then the radiotherapy is stopped;
removing the catheter, along with the motion tracking device, after the radiotherapy is completed.

26. A method for performing extreme hypofractionated image-guided radiotherapy for treating prostate cancer comprising the steps of:
delivering a catheter within a body of the patient, wherein the catheter includes a catheter body having a proximal end and a distal end, the catheter body having a first lumen formed therein, the catheter having a motion tracking device disposed within the first lumen, the motion tracking device having a plurality of transponders that are connected to one another with a connector, the transponders being at fixed distances relative to one another, each transponder emitting a unique signal that is detectable outside the patient's body;
positioning the motion tracking device such that a distalmost transponder of the plurality of transponders at a base of the bladder of the patient and a proximalmost transponder is positioned at a prostatic apex;
detecting any change in the signal emitted from any one of the transponders; and
removing the catheter, along with the motion tracking device, after the medical procedure is completed.

27. A method for performing radiotherapy for treating cervical cancer comprising the steps of:
delivering a catheter within a body of the patient, wherein the catheter includes a catheter body having a proximal end and a distal end, the catheter body having a first lumen formed therein, the catheter having a motion tracking device disposed within the first lumen, the motion tracking device having a plurality of motion tracking elements that are connected to one another with a connector, the motion tracking elements being at fixed distances relative to one another;
anchoring the catheter within the body of the patient by inflating a first catheter balloon within a cervical canal of the patient and inflating a second catheter balloon within a vagina of the patient;
positioning the plurality of motion tracking elements within the first lumen such that the motion tracking elements are positioning within the cervical canal between the first and second catheter balloons;
determining initial locations of the motion tracking elements;
treating target tissue within the cervix;
detecting any change in location coordinates of any one of the motion tracking elements relative to the initial locations during the treatment of the target tissue; and
removing the catheter, along with the motion tracking device, after the treatment of the target tissue is completed.

28. A method for producing anatomical reproducibility and stability of target tissue that is to be treated during a medical procedure comprising the steps of:
delivering a catheter to a location at or proximate to the target tissue within a body of a patient, the catheter including a first lumen which removably receives a first instrument;
inflating a first balloon that is affixed to the catheter to fix the catheter in place at the location;
inflating a second balloon that is associated with a second instrument, the inflation of the second balloon serving to stabilize the target tissue by placing the target tissue under a force; and
using the first instrument to determine an initial position of the target tissue prior to performing the medical procedure, the initial position serving as a baseline from which the anatomical reproducibility and stability of the target tissue are measured.

29. The method of claim 28, wherein the step of using the first instrument to determine the initial position comprises the steps of:
using an imaging device to capture an image of the target tissue along with the first instrument which is formed of a material that is visible in the captured image; and
calculating a location of the first instrument by analyzing the capture image, the location of the first instrument being representative of initial position of the target tissue.

30. The method of claim 28, further including the step of:
detecting, in real-time, any movement of the target tissue during the medical procedure by detecting movement of the first instrument.

31. The method of claim 28, further including the steps of:
removing the first instrument from the first lumen; and
inserting a motion tracking device into the first lumen, the motion tracking device being configured to detect, in real-time, any movement of the target tissue during the medical procedure by detecting movement of the motion tracking device.

32. The method of claim 31, further including the step of:
interrupting the medical procedure when the movement of the target tissue beyond a threshold amount is detected by the motion tracking device.

33. The method of claim 31, further including the step of:
removing the motion tracking device from the lumen during a pause in the medical procedure;
reinserting the first instrument into the first lumen; and
using the imaging device to capture a new image of the target tissue along with the first instrument.

34. The method of claim 33, further including the steps of:
removing the first instrument from the lumen after the new image is captured and during the pause in the medical procedure;
reinserting the motion tracking device into the first lumen;
continuing the medical procedure; and
detecting, in real-time, any movement of the target tissue during the medical procedure by detecting movement of the motion tracking device.

35. The method of claim 28, wherein the catheter is inserted and the first balloon is inflated in one of a urethra and cervical canal and the second instrument is inserted and second balloon is inflated in a rectum of the patient.

36. The method of claim 28, wherein the first instrument includes a plurality of markers that are connected to one another with a connector, the markers being spaced apart at fixed distances relative to one another.

37. The method of claim 36, wherein the plurality of markers comprises three radio-opaque markers that are fixed to the connector.

38. The method of claim 31, wherein the motion tracking device includes motion tracking elements comprising transponders that emit unique signals that can be detected external to the patient's body.
